(19) United States
(12) Patent Application Publication
(10) Pub. No.: US 2008/0260877 A1
(43) Pub. Date: Oct. 23, 2008

(54) Plant extracts and compositions for reducing blood lipid levels

(75) Inventors: Birch Bai, Shanghai (CN); Yi-Cai Fu, Shanghai (CN); XiangGuo Si, Shanghai (CN)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco Inc, d/b/a UNILEVER

(21) Appl. No.: 11/633,807

(22) Filed: Dec. 5, 2006

(30) Foreign Application Priority Data
Dec. 6, 2005 (CN) PCT/CN05/02103
Feb. 9, 2006 (EP) EP06250698

(51) Int. Cl. A61K 36/424; A01H 5/00; A61P 9/00

(52) U.S. Cl. 424/758; 800/298

(57) ABSTRACT

An extract of a plant of the genus Gynostemma is described. The extract is obtainable by a process wherein an aqueous extract is further extracted with n-butanol. The extract has a high level of actives, such as saponins, and/or a low level of impurities. The extract is particularly suitable for use in food or beverage compositions owing to its high purity and clean taste. Such compositions may be used to reduce the blood lipid levels of an individual and provide associated health benefits.

### TECHNICAL FIELD OF THE INVENTION

The present invention relates to plant extracts and compositions for delivering health benefits, particularly in respect of diseases and disorders related to elevated levels of lipids in the blood. In particular the present invention relates to extracts of plants of the genus Gynostemma and food or beverage compositions containing such extracts.

### BACKGROUND OF THE INVENTION

Cardiovascular disease (CVD) is a major cause of death throughout the world. Globally, each year over 11 million people succumb to the two main forms of CVD: coronary heart disease (CHD) and cerebrovascular accident (CA). In developed countries, cardiovascular disease accounts for about 40% of all causes of death; in developing countries this figure is nearly 25%. Results from numerous clinical trials have indicated that interventions designed to modify blood lipid levels significantly reduce the risk of CVD.

Infusions made from various plants have been used as drinks for thousands of years. Such infusions are typically of little nutritional value but are drunk because of their perceived physiological benefits, for example in respect of refreshment and/or health benefits.

Tea is a beverage traditionally made by infusing the dry leaves of the plant Camellia sinensis in boiling water. Tea is (with the exception of water) probably the world's most popular beverage and, in some parts of the world, has traditionally been considered to have health-promoting potential. Recently, extensive laboratory research and epidemiologic studies have shown that compounds present in tea (particularly polyphenolic compounds) may reduce the risk of a variety of illnesses including CVD (see, for example, A. Menotti et al., “Food intake patterns and 25-year mortality from coronary heart disease: cross-cultural correlations in the Seven Countries Study. The Seven Countries Study Research Group.”, Eur. J. Epidemiol., 1999, 15, 507-515). However, although the benefits of tea in respect of reducing the risk of CVD may be apparent at consumption rates as low as three cups per day (U. Peters et al., “Does tea affect cardiovascular disease? A meta-analysis.”, American Journal of Epidemiology, 2001, 154, 495-503), many individuals do not even achieve this modest consumption rate on a long term basis.

Thus there is a need to provide a composition with increased efficacy for delivering health benefits in respect of CVD and other disorders related to blood-lipid levels. In particular there is a need to provide such a composition which is suitable for everyday consumption and/or retains the natural image and palatability of traditional foods and beverages.

Previous attempts have been made to provide palatable beverages with enhanced health benefits by blending tea with other herbs. For example, Chinese patent application CN 1077594 A (H. Chen) discloses a natural healthy tea for preventing and curing cardiovascular and hypertension diseases. The tea is prepared with water extracts of Gynostemma pentaphyllum, Pueraria Lobata and tea leaves. Although the resulting tea is said to maintain the natural appearance and original taste of tea leaves, we have found that extraction of Gynostemma with water yields an extract that contains many inactive impurities and compounds which may produce off-flavours and/or affect the appearance of a food or beverage (e.g. in terms of colour or clarity).

Thus we have recognised that there is a need to provide plant extracts with a higher content of the healthy actives of Gynostemma and/or which do not impart unwanted taste and/or appearance to compositions into which they are incorporated. We have found that extraction of Gynostemma with a specific process provides extracts which meet this need.

### SUMMARY OF THE INVENTION

The present invention provides an extract of a plant of the genus Gynostemma obtainable by a process comprising the steps of:
(i) providing plant material of the plant of the genus Gynos temma;
(ii) contacting the plant material of step (i) with water thereby to form a mixture of insoluble matter and aqueous extract;
(iii) separating the insoluble matter and the aqueous extract of step (ii);
(iv) contacting the aqueous extract of step (iii) with n-butanol to form a mixture comprising insoluble matter and butanolic extract;
(v) separating the butanolic extract and the insoluble matter of step (iv); and
(vi) recovering the extract of a plant of the genus Gynostemma from the butanolic extract.

The aqueous extract of steps (ii) and (iii) is found to contain all of the major saponins of Gynostemma. However, the aqueous extract usually contains undesirable impurities such as saccharides and chlorophyll. The further treatment of the aqueous extract with n-butanol results in an extract wherein a large proportion of these impurities is removed.

The present invention also provides compositions, such as foods or beverages, comprising the Gynostemma extract. The compositions preferably comprise tea solids and/or an extract of a plant of the genus Pueraria. Such compositions have been found to be effective at lowering blood lipid levels and thus a second aspect the present invention provides use of the compositions for reducing the blood lipid level of an individual; and/or for providing associated health benefits.

The present invention also provides the process by which the extract of a plant of the genus Gynostemma is obtained.

### DETAILED DESCRIPTION OF THE INVENTION

### Extract of a Plant of the Genus Gynostemma

Plants of the genus Gynostemma are herbs of the family Cucurbitaceae. Gynostemma pentaphyllum is a perennial, deciduous, creeping herb that grows in the south eastern provinces of China, as far north as northern Anhui, and is found in lesser quantities in South Korea and Japan. It has many common names including “fiveleaf gynostemma”, “jiaogulan” and “amachazuru”. There are at least thirteen varieties under this genus, of which eleven are grown in China.

Gynostemma is rich in saponins, with a content of gypenosides of more than 5.0% by weight. Without wishing to be bound by theory, we believe that it is these saponins that are primarily responsible for the health benefits provided by Gynostemma. The major saponins of Gynostemma pentaphyllum are gypenoside XLII, gypenoside LVI, gypenoside XLIII, gypenoside XLVI, gypenoside XLIV, gypenoside Rd, gypenoside LXXIV, and gypenoside LXXVII. The chemical structures of these gypenosides are given in formulae A to H below.

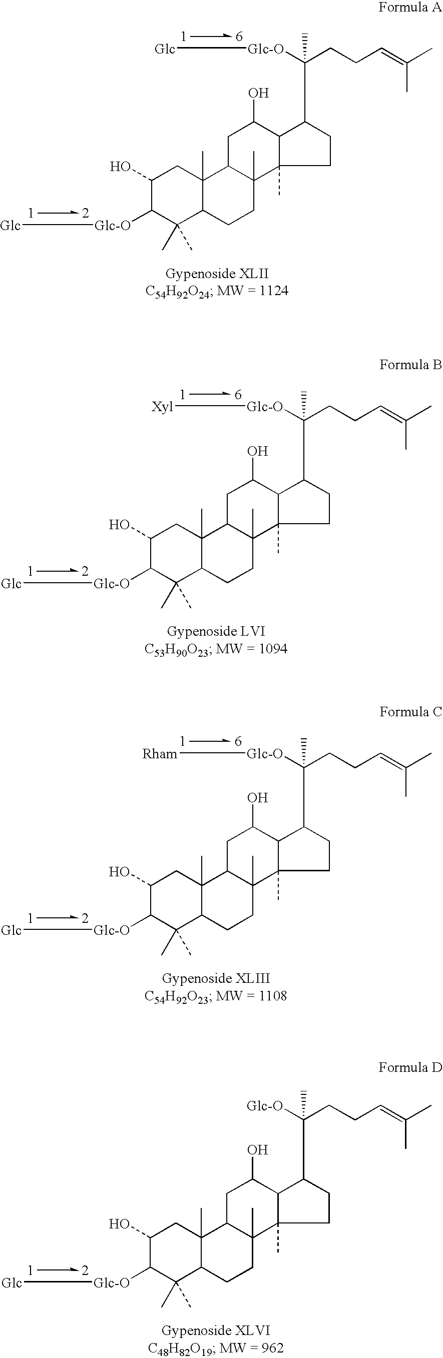

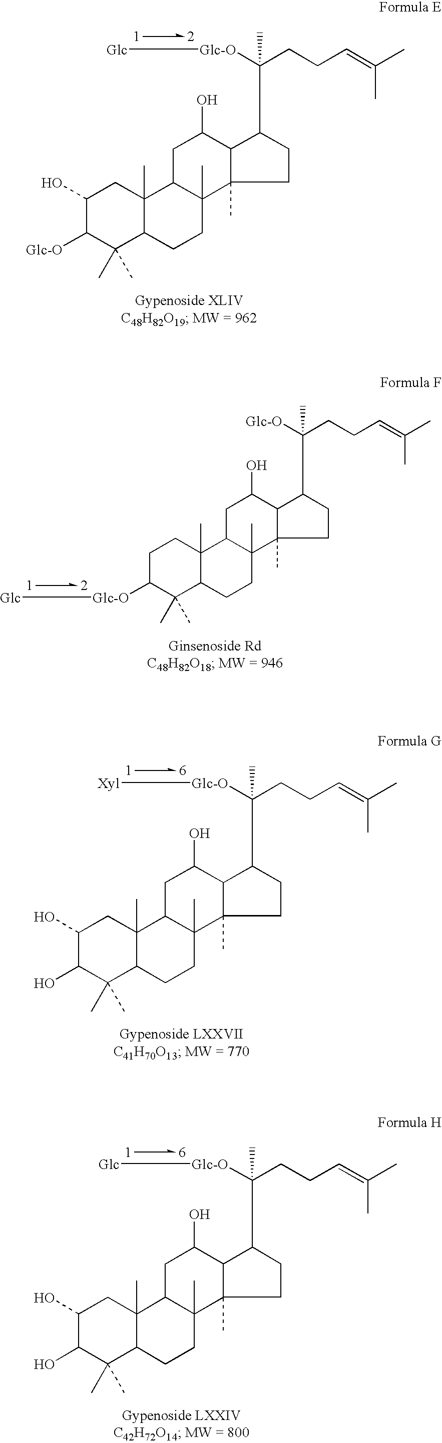

The term “extract of a plant of the genus Gynostemma” as used herein refers to solids extractable by treating material of the plant with a solvent. The extract of a plant of the genus gynostemma is obtained and/or obtainable by a process comprising the steps of:
(i) providing plant material of the plant of the genus Gynostemma;
(ii) contacting the plant material of step (i) with water thereby to form a mixture of insoluble matter and aqueous extract; and
(iii) separating the insoluble matter and the aqueous extract of step (ii).

Preferably the plant material and water are contacted in step (ii) in a weight ratio of from 1:1 to 1:1000, more preferably from 1:2 to 1:100 and most preferably from 1:5 to 1:50. The water may be contacted with the plant material in a single dose, or the water may be split into a plurality of doses with each dose being contacted sequentially with the plant material and the resulting aqueous extracts being pooled.

Preferably step (ii) comprises contacting the plant material with boiling water for a duration of at least 30 minutes, more preferably for a duration of between 30 minutes and 10 hours, optimally between 1 hour and 5 hours.

The aqueous extract so obtained usually contains all of the major saponins. However, the aqueous extract usually also contains undesirable impurities such as saccharides and chlorophyll. We have found that by further treating the aqueous extract with n-butanol, a large proportion of these impurities can be removed in a simple manner. Thus the process comprises the additional steps of:
(iv) contacting the aqueous extract with n-butanol to form a mixture comprising insoluble matter and butanolic extract; and
(v) separating the butanolic extract and the insoluble matter of step (iv).

The extract of the plant of the genus Gynostemma is then recovered from the butanolic extract. This is usually achieved by removing butanol from the extract, for example by evaporation, and the resulting butanol may be recycled, for example for use in a repeat of step (iv) of the process.

Preferably the aqueous extract is concentrated, for example by drying, prior to step (iv) in order that the extraction with butanol is most efficient.

Preferably the aqueous extract and n-butanol are contacted in step (iv) in a weight ratio of from 2:1 to 1:100, more preferably from 1:1 to 1:50 and most preferably from 1:2 to 1:10. The aqueous extract may be contacted with the butanol in a single dose, or the butanol may be split into a plurality of doses with each dose being contacted sequentially with the aqueous extract and the resulting butanolic extracts being pooled.

Preferably the aqueous extract and n-butanol are contacted in step (iv) at a temperature of between 5 and 40° C., more preferably between 10 and 30° C. The duration of contact is preferably from 30 minutes to 15 hours, more preferably from 1 hour to 10 hours. A strain of Gynostemma pentaphyllum known as “Variety 201”, or “sweet gynostemma” has been developed in Japan. We have found that this variety is particularly suitable for use in the present invention as it is particularly efficacious at reducing blood lipid levels and has a minimum impact on taste when employed in a food or beverage.

The extract of a plant of the genus Gynostemma of this invention is preferably rich in saponins. The saponins are predominantly found in the leaves and stem of the plant and so it is preferred that the extract is an extract of the leaves and/or stem of a plant of the genus Gynostemma, most preferably of the leaves.

In a preferred embodiment the extract of a plant of the genus Gynostemma comprises a saponin mixture of gypenoside XLII, gypenoside LVI, gypenoside XLIII, gypenoside XLVI, gypenoside XLIV, gypenoside Rd, gypenoside LXXIV and gypenoside LXXVII. The plant extract preferably comprises the saponin mixture in an amount of at least 5% by weight of the extract, more preferably at least 15% and most preferably from 30 to 100%.

### Compositions

The Gynostemma extracts of the invention are particularly suitable for use in food or beverage compositions owing to the low level of impurities.

In a particularly preferred embodiment, the Gynostemma extract is included in a composition which comprises other actives known to provide health benefits related to reduced levels of blood lipids. In particular, compositions are preferred which comprise tea solids and/or an extract of a plant of the genus Pueraria.

The amount of the extract of a plant of the genus Gynostemma present in compositions of the invention is preferably from 0.1 to 50% by dry weight of the composition, more preferably from 1 to 40% and optimally from 4 to 35%.

### Tea Solids

As used herein, the term “tea solids” refers to dry material from the leaves of Camellia sinensis var. sinensis and/or Camellia sinensis var. assamica. The leaves may have been subjected to a so-called “fermentation” step wherein they are oxidised by certain endogenous enzymes that are released during the early stages of “black tea” manufacture. This oxidation may even be supplemented by the action of exogenous enzymes such as oxidases, laccases and peroxidases. Alternatively the leaves may have been partially fermented (“oolong” tea) or substantially unfermented (“green tea”).

The tea solids may be water-soluble (e.g. in boiling water) tea solids and/or water-insoluble tea solids. In particular the tea solids may be selected from tea leaf, tea extract (e.g. obtained by contacting tea leaf material with water) and mixtures thereof.

Owing to the recognised efficacy of tea polyphenols in combating CVD, it is especially preferred that the tea solids comprise a polyphenolic compound selected from the group consisting of catechins, theaflavins, thearubigins, and mixtures thereof. Such polyphenolic compounds are described in detail in Chapter 17 of “TEA: Cultivation to Consumption” (K. C. Wilson and M. N. Clifford (Eds), 1992, Chapman & Hall, London). Preferably the composition comprises at least 5 mg of the polyphenolic compound, more preferably from 10 to 2000 mg and optimally from 20 to 500 mg. Although it is preferred that the polyphenolic compound is present as part of the tea solids, the composition may alternatively or additionally comprise a polyphenolic compound as described above but derived from a non-tea source, such as a synthetic polyphenolic compound.

Green tea is especially rich in catechins and so it is preferred that the tea solids comprise green tea solids.

The amount of tea solids will depend on the form of the composition. However, in order to provide maximum taste and health benefits, it is preferred that the tea solids are present in an amount of from 10 to 98% by dry weight of the composition, more preferably from 25 to 95% and optimally from 45 to 90%.

### Pueraria

Plants of the genus Pueraria are vines of the pea family Fabaceae. There are several species of Pueraria throughout the world that are variously referred to as “kudzu”. Pueraria montana var. lobata (Willd.) Maesen & S. Almeida is a climbing, semi-woody, perennial vine native to East Asia but which has also become widely established elsewhere in the world, such as the south-eastern United States. Its common names include “lobed kudzuvine”.

Pueraria is rich in flavonoids. Without wishing to be bound by theory, we believe that it is these flavonoids that are primarily responsible for the health benefits provided by Pueraria. The major flavonoids of Pueraria montana are puerarin, 3′-methoxy-puerarin, 3-methoxy-6″-O-acetylpuerarin, 6″-O-acetylpuerarin and daidzein. The chemical structures of these flavonoids are given in formulae I to M below.

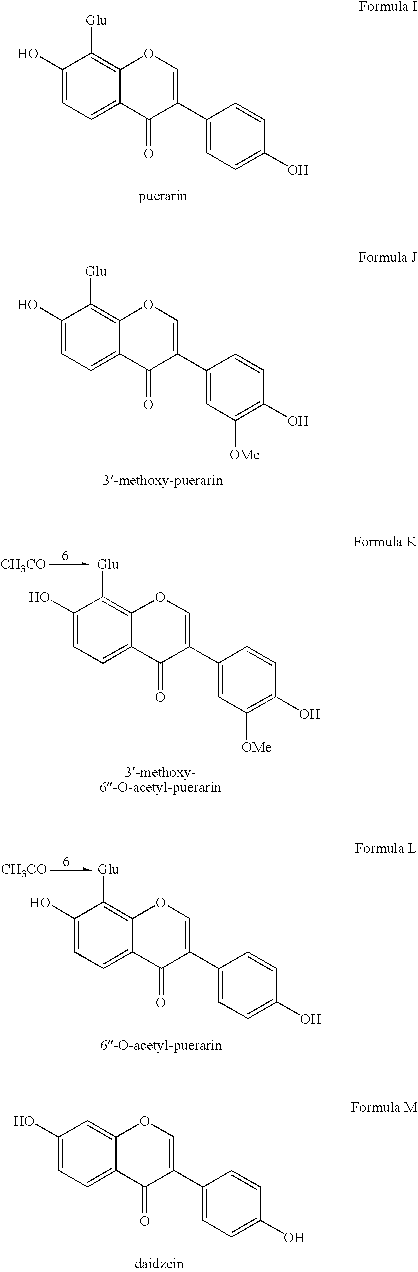

Compositions according to the present invention preferably comprise an extract of a plant of the genus Pueraria. The term “extract of a plant of the genus Pueraria” as used herein refers to solids extractable by treating material of the plant with a solvent. The preferred extracts are those soluble in boiling water and include extracts obtainable by treating plant material with polar solvents such as water, lower alcohols (e.g., C₁-C₅ alcohols) and mixtures thereof. In a preferred embodiment the extract of a plant of the genus Pueraria is obtained and/or obtainable by a process comprising the steps of:
(I) providing plant material of the plant of the genus Pueraria;
(II) contacting the plant material of step (I) with ethanol to form a mixture of insoluble matter and ethanolic extract; and
(III) separating the ethanolic extract and the insoluble matter of step (II).

The ethanolic extract so obtained usually contains all of the major flavonoids. The ethanol may be directly removed to yield an extract suitable for use in this invention. However, the ethanolic extract usually contains undesirable impurities such as saccharides and chlorophyll. We have found that by further treating the ethanolic extract with n-butanol, a large proportion of these impurities can be removed in a simple manner. Thus in an especially preferred embodiment, the process comprises the additional steps of:
(IV) contacting the ethanolic extract with n-butanol to form a mixture comprising insoluble matter and butanolic extract; and
(V) separating the butanolic extract and the insoluble matter of step (IV).

The butanol is usually then removed from the extract and may be recycled. In a preferred embodiment the butanolic extract is further purified by re-crystallizing from aqueous acetic acid.

The extract of a plant of the genus Pueraria is preferably rich in flavonoids. The flavonoids are predominantly found in the roots of the plant and so it is preferred that the extract is an extract of the roots of a plant of the genus Pueraria.

In a preferred embodiment the extract of a plant of the genus Pueraria comprises a flavonoid selected from the group consisting of puerarin, daidzein, 3′-methoxy-puerarin, 3′-methoxy-6″-O-acetylpuerarin, 6″-O-acetylpuerarin, and mixtures thereof. The plant extract preferably comprises the flavonoid in an amount of at least 15% by weight of the extract, more preferably at least 30% and most preferably from 50 to 100%. Although it is preferred that the flavonoid is present as part of the plant extract, the composition may alternatively or additionally comprise a flavonoid as described above but derived from a non-Pueraria source, such as a synthetic flavonoid.

The amount of the extract of a plant of the genus Pueraria present in the composition is preferably from 0.1 to 50% by dry weight of the composition, more preferably from 1 to 40% and optimally from 4 to 35%.

### Product Form and Packaging

Compositions of the present invention may be in any form but are preferably in a form suitable for direct oral administration and/or consumption. Suitable forms include capsules, pills, tablets, syrups, liquids, powders, granules and gels. In a preferred embodiment the composition is a food and in a most preferred embodiment it is a beverage or a beverage precursor.

### Beverage and Beverage Precursor

As used herein the term “beverage” refers to a substantially aqueous drinkable composition suitable for human consumption. The beverage will typically comprise at least 70% water, more preferably at least 80%, optimally between 85 and 99% by weight of the beverage. The total amount of tea solids (if present), extract of Pueraria (if present) and extract of Gynostemma will typically be from 0.001 to 5%, more preferably from 0.01 to 3% and most preferably from 0.1 to 1% by weight of the beverage. Any tea solids in the beverage preferably consist of soluble tea solids. The beverage may contain other optional components such as a chelator, colorant, preservative (e.g., potassium sorbate, sodium benzoate or a mixture thereof), flavour, vitamin, sweetener (e.g, corn syrup, sucrose or a mixture thereof), fruit juice, surfactant (e.g, sorbitan monolaurate, sorbitan monopalmitate or a mixture thereof), acidulant, non-fat milk solids, fat (e.g. milk fat, vegetable fat or a mixture thereof) or mixtures thereof. When employed, such optional components collectively make up less than 25.0% by weight of the total weight of the beverage composition, typically between 0.001% and 10%. The beverage may be still or carbonated.

As used herein, the term “beverage precursor” is defined as a fabricated composition suitable for preparing a beverage. The beverage precursor may be in various forms including a liquid concentrate or a powdered or granulated solid. To provide maximum storage stability, the beverage precursor will typically contain less than 50% water, more preferably less than 25% water and optimally between 0.1 and 10% water by weight of the beverage precursor. In an especially preferred embodiment the beverage precursor comprises tea leaf optionally combined with powdered or granulated soluble tea solids. The tea leaf may be loose but is preferably packaged in an infusion package such as a tea bag.

The present invention also provides a method of preparing a beverage, the method comprising the steps of providing the beverage precursor of the invention and then contacting the precursor or a part thereof with an aqueous medium thereby to form the beverage. The aqueous medium may be any edible liquid but is preferably selected from water and/or milk. Preferably also, the aqueous medium is hot and has a temperature of at least 30° C., preferably at least 60° C., more preferably between 75 and 100° C. In an alternative embodiment, the aqueous medium is cold and has a temperature of less than 30° C., preferably less than 15° C., more preferably between 1 and 100° C.

### Use of the Compositions

A composition of the invention may simply be enjoyed as a food or beverage but in a preferred embodiment it is used as a medicament. In particular, the composition may be used in a method for reducing the blood lipid level of an individual and for treating and/or preventing related disorders such as cardiovascular disease and obesity. The composition may also be used in the control of the bodyweight of an individual. The composition is typically administered orally to the individual.

The composition may also be used in the manufacture of a medicament for reducing the blood lipid level of an individual; and/or for treating or preventing cardiovascular disease; and/or for treating or preventing obesity; and/or to assist in the control of the bodyweight of an individual.

### EXAMPLES

### Example 1

### Preparation of Extract of Gynostemma

1 part by weight of the dry leaves (supplied by Hubei Shientang Pharmaceutical Co.) of plants of the sweet variety of fiveleaf gynostemma (Gynostemma pentaphyllum) was extracted 3 times in 10 parts of boiling water, 1 hour per time. The resulting 30 parts of aqueous extract were then pooled and the pooled aqueous extract filtered and the water evaporated to give a concentrated aqueous extract with a density of 1.15. The concentrated aqueous extract was then dispersed in 0.8 parts of water-saturated n-butanol at room temperature (−20° C.), and allowed to separate for 1 hour. The separated aqueous phase was then re-dispersed in a fresh portion of n-butanol. This butanol extraction was executed a total of 5 times and the resulting butanolic extracts pooled. The n-butanol was then evaporated from the pooled butanolic extract under vacuum and the resulting dense liquid spray-dried to yield the extract of the plant of the genus Gynostemma. The extract was then analysed by HPLC with the following conditions:

| | |
|---|---|
| Column: | Hypersil ™ ODS, 4.6 mm × 250 mm, 5 μm. |
| Detection | 203 nm. |
| wavelength: | |
| Temperature: | 40° C. |
| Flow rate: | 1.0 ml/min. |
| Solvent: | A - 100% MeCN; B - H₂O. |
| Gradient: | 0-30 min of 30-37.5% A and 70-62.5% B (linear gradient); |
| | 31-38 min of 45% A and 55% B (isocratic); |
| | 38-48 min of 45-50% A and 55-50% B (linear gradient); |
| | 48-55 min of 50-100% A and 50-0% B (wash); |
| | 55-65 min of 100-30% A and 0-70% B (linear gradient). |

The analysis showed that the extract contained a total of 34% by dry weight of gypenoside XLII, gypenoside LVI, gypenoside XLIII, gypenoside XLVI, gypenoside XLIV, gypenoside Rd, gypenoside LXXIV and gypenoside LXXVII.

### Example 2

### Preparation of Extract of Pueraria

1 part by weight of dried roots (supplied by Ankang Heye Maidisen Plant Pharmaceutical Co.) of lobed kudzuvine (Pueraria Montana var. lobata (Willd.)) was extracted 3 times (1 hour per time) in 10 parts of 60% ethanol in water at 100° C. The resulting 30 parts of ethanolic extract were then pooled and the solvent evaporated from the pooled ethanolic extract to give a crude residue. This residue was then dispersed in 10 parts n-butanol and any insoluble matter discarded. The butanol was then removed from the butanolic extract and the resulting residue recrystallized from 10% acetic acid (aqueous) to yield the final white powdery extract of Pueraria.

The extract was then analysed by HPLC with the following conditions:

| | |
|---|---|
| Column: | Hypersil ™ ODS, 4.6 mm × 200 mm, 5 μm. |
| Detection wavelength: | 255 nm. |
| Temperature: | 25° C. |
| Flow rate: | 1.0 ml/min. |
| Solvent: | A - 100% MeCN; B - H₂O. |
| Mobile phase: | 0 min - 5% A; |
| | 35 min - 50% A; |
| | 40 min - 5% A. |

The analysis showed that the extract contained a total of 91% by dry weight of puerarin, daidzein, 3′-methoxy-puerarin, 3′-methoxy-6″-O-acetylpuerarin and 6″-O-acetylpuerarin.

### Example 3

### Animal Efficacy Studies

Animal studies were performed to assess the efficacy of various tea-based beverages on lowering blood lipid levels. The protocol for the animal efficacy studies (including the statistical analysis of the results) was as prescribed in “The Assessment of Assisting Blood Lipids Reduction Function”, Technical Standards for Testing & Assessment of Health Food—2003 version, China National Health Inspection, Ministry of Health, People's Republic of China, February 2003.

### Materials and Methods

### Rats:

Fifty SD rats, each with a body weight in the range 180 g to 200 g were supplied by Shanghai Xipuer-Bikai Exp Animal Co.

### Tea-Based Beverages:

Beverages made from infusing tea bags of two formulations were tested. Tea bags of Formulation 1 contained beverage precursor solids consisting of 35% by weight of the fiveleaf gynostemma extract of Example 1, 10% by weight of the lobed kudzuvine extract of Example 2 and 55% by weight green tea leaf (Liptonl brand green tea). Tea bags of Formulation 2 contained beverage precursor solids consisting of 35% by weight of the fiveleaf gynostemma extract of Example 1 and 65% by weight of green tea leaf (Lipton green tea)

Infusions of Formulation 1 were prepared in three dosage strengths 0.333 g, 0.666 g and 2 g beverage precursor solids per kg bodyweight. Infusions of Formulation 2 were made to a single strength of 0.666 g per kg bodyweight. The infusions of a given dosage strength were prepared as follows for each rat:
The rat was weighed to give a bodyweight (e.g. 200 g).
A tea bag was then prepared containing a total amount of beverage precursor solids equal to the product of the bodyweight multiplied by the dosage strength (e.g. 200 g×2 g kg⁻¹=0.4 g of beverage precursor solids).
The tea bag was then infused in a 10 fold excess (e.g. 4 g) of water at 85° C. for 30 minutes.
The tea bag was then infused in a fresh portion of water in an identical manner.
The teabag was then discarded and the two portions of infusion pooled (e.g. to give a total 8 g infusion).
The pooled infusion was then condensed to the required gavage dose volume (e.g. for a gavage dose of 1 ml 100 g⁻¹ bodyweight this would be 2 ml).

### Determination of Total Cholesterol (TC):

A COD-POD method was used to determine the total cholesterol concentration in the blood of each rat. This involved decomposing cholesterol ester by cholesterol esterase (CE) into free cholesterol and fatty acid. The free cholesterol is then transformed, due to the effect of cholesterol oxidase (CO), into steroid, and at the same time, hydrogen peroxide (H₂O₂) is produced. Peroxidase (POD) causes hydrogen peroxide to induce oxidation and condensation of N-(2-hydroxy-3-sulfopropyl)-3,5 dimethoxyaniline sodium salt (HDAOS) and 4-aminoantipyrine to produce a purple pigment. The increment in this pigment is determined at a wavelength of 500 nm to obtain a concentration of total cholesterol.

### Determination of Triglycerides (TG):

A GK-GPO method was used to determine the concentration of triglycerides in the blood of each rat. Firstly, ascorbic acid was removed by ascorbate oxidase (AOD). Next, free glycerol in the sample was decomposed into hydrogen peroxide by glycerol kinase (GK) and glycerol-3-oxidase phosphate (GPO) in 2 chemical reactions. Catalase was added at the same time as hydrogen peroxide generation to remove the influence of free glycerol. In the second reaction, lipoprotein lipase (LPL) and 4-aminoantipyine (4AA) are added to induce reaction. As a result of this reaction, N-(2-hydroxy-3-sulfopropyl)-3,5-dimethoxyanilin sodium salt (HDAOS) and 4AA quantitatively cause oxidizing condensation of hydrogen peroxide to produce a purple quinone dye after adding POD. The increased quantity of this dye is determined at wavelengths of 550 nm so as to obtain a TG concentration.

Determination of High Density Lipoprotein—cholesterol (HDL-c): Polyanions and polyethylene glycol (PEG)-6000 were used to selectively aggregate and render insoluble the lower density lipoproteins (LDL) and very lower density lipoproteins (VLDL), leaving HDL in solution. The insoluble lipoproteins were then sedimented by low-speed centrifugation. The COD-POD method was then used to determine the HDL-c level in the supernatant.

### Experimental Design

### Test Groups:

The rats were divided into five test groups of ten rats each. The groups were labelled according to the beverage with which the group was to be administered as set out in Table 1.

TABLE 1
Test Groups

| Group | Beverage |
|---|---|
| Control | Distilled water |
| Low | Infusion of 0.333 g kg⁻¹ Formulation 1 |
| Medium | Infusion of 0.666 g kg⁻¹ Formulation 1 |
| High | Infusion of 2.0 g kg⁻¹ Formulation 1 |
| Fiveleaf | Infusion of 0.666 g kg⁻¹ Formulation 2 |

### Conditioning:

The rats were adapted to the new environment under fundamental feeding for 5 days. The body weight of each rat was then measured and its blood sampled through the venous plexus of the orbital sinus and analysed for TC, TG and HDL-c levels. Each of these measurements was then averaged over the ten rats of each group and the resulting mean values are given in Table 2.

TABLE 2
Initial Condition of the Test Groups

| | TC | TG | HDL-c | Weight |
|---|---|---|---|---|
| Group | (mmol/l) | (mmol/l) | (mmol/l) | (g) |
| Control | 2.58 | 1.33 | 1.67 | 186.2 |
| Low | 2.60 | 1.32 | 1.66 | 184.1 |
| Middle | 2.59 | 1.36 | 1.71 | 186.7 |
| High | 2.59 | 1.25 | 1.71 | 183.9 |
| Fiveleaf | 2.59 | 1.02* | 1.70 | 185.0 |

*Significantly different from control.

It can be seen that, apart from a slight difference in TG level for the Fiveleaf group, there was no significant difference in initial condition between the control and experiment groups.

### Treatment:

Each test group was allowed free access to high fat feed and water. Each rat was administered with a daily intragastric (i.g.) gavage dose of 1 ml/100 g of body weight of a beverage as indicated in Table 1. This daily treatment was continued for a total of 30 days. At the end of the 30 days, each rat was weighed and its blood sampled through the venous plexus of the orbital sinus and analysed for TC, TG and HDL-c level. Each of these measurements was then averaged over the ten rats of each group and the resulting mean values are given in Table 3.

TABLE 3
Condition of Test Groups After 30 days of Treatment

| | TC | TG | HDL-c | Weight |
|---|---|---|---|---|
| Group | (mmol/l) | (mmol/l) | (mmol/l) | (g) |
| Control | 3.08 | 2.47 | 1.08 | 353.9 |
| Low | 2.87 | 1.64* | 1.11 | 339.1 |
| Middle | 2.74* | 1.27* | 1.02 | 358.0 |
| High | 2.64* | 1.17* | 1.03 | 322.2* |
| Fiveleaf | 2.78 | 1.70* | 1.17 | 355.6 |

*Significantly different from control.

These results show that treatment with a composition comprising tea and a Gynostemma extract of the invention (‘Fiveleaf’ group) can significantly decrease blood levels of triglycerides compared with treatment with the control. Furthermore, larger reductions in triglycerides can be achieved by treatment with a composition comprising tea, Gynostemma and Pueraria (‘Low’, ‘Middle’ and ‘High’ groups). In addition, treatment with compositions comprising tea, Gynostemma and Pueraria can significantly decrease the blood levels of cholesterol (‘Middle’ and ‘High’ groups) and body weight (‘High’ group) compared with treatment with the control.

1. An extract of a plant of the genus Gynostmma obtainable by a process comprising the steps of:
(i) providing plant material of the plant of the genus Gynostemma;
(ii) contacting the plant material of step (i) with water thereby to form a mixture of insoluble matter and aqueous extract;
(iii) separating the insoluble matter and the aqueous extract of step (ii)
(iv) contacting the aqueous extract of step (iii) with n-butanol to form a mixture comprising insoluble matter and butanolic extract;
(v) separating the butanolic extract and the insoluble matter of step (iv); and
(vi) recovering the extract of a plant of the genus Gynostemma from the butanolic extract.

2. An extract of a plant of the genus Gynostemma according to claim 1 wherein step (ii) comprises contacting the plant material with boiling water for a duration of at least 30 minutes.

3. An extract of a plant of the genus Gynostemma according to claim 1 comprising a seponin mixtures of gypenoside XLII, gypenoside LVI, gypenoside XLIII, gypenoside XLVI, gypenoside XLIV, gypenoside Rd, gypenoside LXXIV and gypenoside LXXVII.

4. An extract of a plant of the genus Gynostemma according to claim 3 wherein the extract comprises the saponin mixture in an amount of at least 15% by weight of the extract.

5. An extract of a plant of the genus Gynostemma according to claim 1 wherein the plant of the genus Gynostemma is of the species Gynostemma pentaphyllum.

6. An extract of a plant of the genus Gynostemma according to claim 5 wherein the Gynostemma pentaphyllum is of the sweet variety.

7. A food or beverage composition comprising an extract of a plant of the genus Gynostemma according to claim 1.

8. A composition comprising:
(a) tea solids;
(b) extract of a plant of the genus Gynostemma according to claim 1.

9. A composition according to claim 8 wherein the tea solids comprise a polyphenolic compound selected from the group consisting of catechins, theaflavins, thearubigins, and mixtures thereof.

10. A composition according to claim 7 wherein the composition additionally comprises extract of a plant of the genus Pueraria.

11. A composition according to claim 10 wherein the plant of the genus Pueraria is of the species Pueraria Montana.

12. A composition according to claim 10 herein the extract of a plant of the genus Pueraria comprises a flavonoid selected from the group consisting of puerarin, daidzein, 3′-methoxy-puerarin, 3′-methoxy-6″-O-acetylpuerarin, 6″-O-acetylpuerarin, and mixtures thereof.

13. A composition according to claim 8 wherein the composition is a beverage or beverage precursor composition.

14. A composition according to claim 13 wherein the composition is a beverage precursor composition and wherein the tea solids comprise tea leaf.

15. A packaged beverage precursor comprising an infusion package containing a composition according to claim 8.

16. A method for reducing the blood lipid level of an individual; and/or for treating or preventing cardiovascular disease; and/or for treating or preventing obesity; and/or to assist in the control of the bodyweight of an individual; the method comprising the step of administering to the individual a composition according claim 7.

17. A method according to claim 16 wherein the composition is administered orally.

18. The composition according to claim 7 for use as a medicament.

19. A process for manufacturing an extract of a plant of the genus Gynostmma, the process comprising the steps of:
(i) providing plant material of the plant of the genus Gynostemma;
(ii) contacting the plant material of step (i) with water thereby to form a mixture of insoluble matter and aqueous extract;
(iii) separating the insoluble matter and the aqueous extract of step (ii)
(iv) contacting the aqueous extract of step (iii) with n-butanol to form a mixture comprising insoluble matter and butanolic extract;
(v) separating the butanolic extract and the insoluble matter of step (iv); and
(vi) recovering the extract of a plant of the genus Gynostemma from the butanolic extract.
